(19) United States
(12) Patent Application Publication
(10) Pub. No.: US 2016/0051410 A1
(43) Pub. Date: Feb. 25, 2016

(54) WATERPROOF GOGGLE

(71) Applicant: QBAS CO., LTD., Taipei (TW)

(72) Inventor: Chih-Cheng SHIUE, Taipei (TW)

(73) Assignee: QBAS CO., LTD., Taipei (TW)

(21) Appl. No.: 14/830,465

(22) Filed: Aug. 19, 2015

(30) Foreign Application Priority Data
Aug. 19, 2014 (TW) 103214798

(51) Int. Cl. A61F 9/02

(52) U.S. Cl. CPC A61F 9/029; A61F 9/026

(57) ABSTRACT

A waterproof goggle is provided which includes a frame, a lens and a supporting structure. The frame has an inner ring surface, an outer ring surface which is opposite to the inner ring surface and a plurality of convex engaging teeth which are disposed on the inner ring surface. The lens is surrounded by the inner ring surface and has a non-porous rim portion which is clamped by the convex engaging teeth to affix the lens into the frame. The supporting structure has a covering portion and a supporting portion which extends backwards from the covering portion. The covering portion encapsulates the inner ring surface, the convex engaging teeth and the non-porous rim portion to render a gap between the inner ring surface and the non-porous rim portion waterproof

### CROSS-REFERENCES TO RELATED APPLICATIONS

This application claims priority to Taiwan Patent Application No. 103214798 filed on Aug. 19, 2014, which is hereby incorporated herein by reference in its entirety.

### BACKGROUND OF THE INVENTION

1. Field of the Invention

The present invention provides a pair of glasses; in particular, a pair of glasses that are waterproof goggles.

2. Descriptions of the Related Art

Waterproof goggles are used during waterborne or underwater activities to prevent water or dust from getting into direct contact with the eyes. To satisfy this demand for waterproofing and dustproofing, connections between members of the waterproof goggle need to be sufficiently tight and close so that water and dust cannot pass therethrough.

In particular, the close connections between the members of the waterproof goggle are achieved by using a covering material, i.e., the covering material encapsulates a frame and the lens of the waterproof goggle so that no air gap exists between the frame and the lens.

However, conventional waterproof goggles require the use of a relatively large quantity of covering materials because a plurality of apertures needs to be formed at the rim of the lens. Particularly, as shown in FIGS. 4A to 4C, the lens 91 of a conventional waterproof goggle 9 has a plurality of apertures 911. These apertures 911 are distributed around the periphery of the lens 91 and arranged in the form of a ring. A covering material 92 will pass through and block these apertures 911 so that water or dust cannot pass through these apertures 911. The covering material 92 that blocks the apertures 911 pulls the lens 91 tight to fix the lens 91 into the frame. To block the apertures 911, a relatively large quantity of covering materials 92 are required.

Because a relatively large quantity of covering materials is required, the material cost will inevitably be increased and the frame of the waterproof goggle will become thick. A thick frame is not appealing in appearance and further increases the resistance to the flow of water.

Accordingly, a need exists in the art to provide a waterproof goggle which can improve at least one of the aforesaid drawbacks.

### SUMMARY OF THE INVENTION

An objective of the present invention is to provide a waterproof goggle, which can reduce the use of the covering material and make the frame thinner.

To achieve the aforesaid objective, a waterproof goggle disclosed in the present invention comprises the following: at least one frame, with a ring structure that has an outer ring surface, an inner ring surface and a plurality of convex engaging teeth, wherein the inner ring surface is spaced apart from the outer ring surface and the convex engaging teeth are disposed on the inner ring surface; at least one lens, which is surrounded by the inner ring surface of the frame and that has a non-porous rim portion clamped by the convex engaging teeth to affix the lens into the frame; and at least one supporting structure, with a covering portion and a supporting portion which extends backwards from the covering portion, wherein the covering portion encapsulates the inner ring surface, the convex engaging teeth of the frame and the non-porous rim portion of the lens to render a gap between the inner ring surface and the non-porous rim portion waterproof.

Thereby, the waterproof goggle disclosed in the present invention can at least have the following technical effect: the lens thereof can remain fixed without being perforated so that the range of the lens to be covered by the covering material is reduced.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a perspective assembly view of a waterproof goggle according to the preferred embodiment of the present invention;

FIG. 2 is a perspective exploded view of the waterproof goggle according to the preferred embodiment of the present invention;

FIGS. 3A and 3B are cross-sectional perspective views of the waterproof goggle (taken along section lines AA and BB respectively of FIG. 1) according to the preferred embodiment of the present invention; and

FIGS. 4A to 4C are schematic views of a conventional waterproof goggle.

### DESCRIPTION OF THE PREFERRED EMBODIMENT

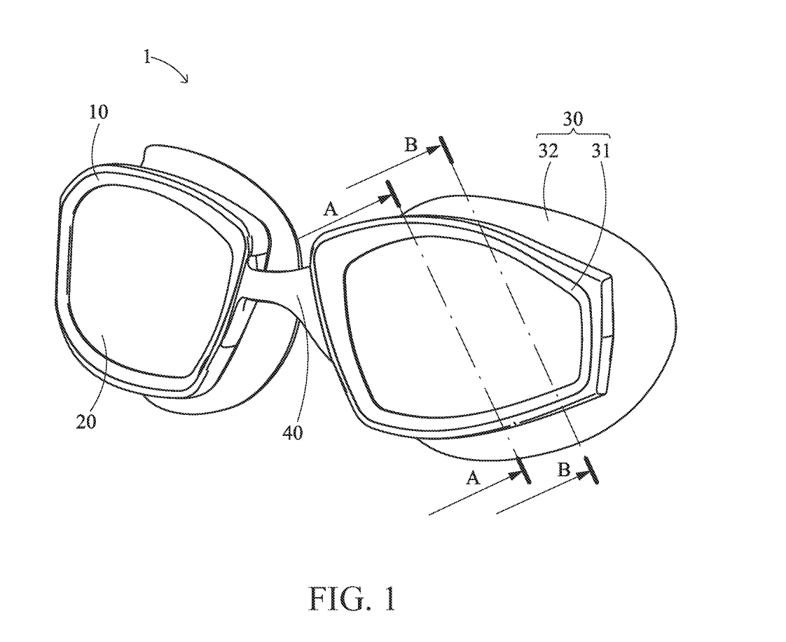

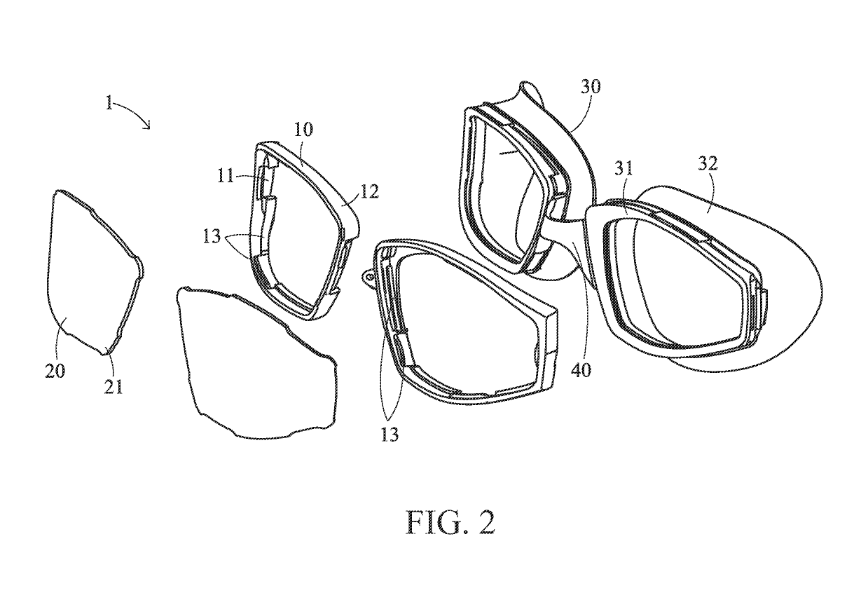

FIGS. 1 and 2 are shown for a perspective assembly view and a perspective exploded view of a waterproof goggle according to the preferred embodiment of the present invention. A waterproof goggle 1 comprises two frames 10, two lenses 20, two supporting structures 30 and a connection piece 40. The technical contents of the aforesaid elements will be described in sequence as follows.

Each of the frames 10 is of a ring structure and comprises an inner ring surface 11 and an outer ring surface 12 that are spaced apart from each other. That is, the inner ring surface 11 and the outer ring surface 12 are not in direct connection with each other but are connected with each other indirectly through a front side surface or a rear side surface of the frame 10. The distance between the inner ring surface 11 and the outer ring surface 12 may be defined as the thickness of the frame 10.

The frame 10 further has a plurality of convex engaging teeth 13 disposed on the inner ring surface 11, and the convex engaging teeth 13 may be distributed anywhere on the inner ring surface 11 and may be formed integrally with the inner ring surface 11. The convex engaging teeth 13 are used to clamp the lens 20 to affix the lens 20 into the frame 10. Preferably, any two neighboring ones of the convex engaging teeth 13 are staggered in a front-back direction; that is, two neighboring convex engaging teeth 13 are spaced apart from each other in the assembling direction of the lens 20 and the frame 10.

Thus, a gap is formed between two neighboring convex engaging teeth 13 in the assembling direction so that the lens 20 can be placed therein. The gap may correspond to the thickness of the lens 20. In addition to being disposed in a staggered front-back direction, the convex engaging teeth 13 may have different profiles to suitably clamp the lens 20 in accordance with the change of the outline of the lens 20.

The lens 20 has a non-porous rim portion 21, i.e., the rim portion of the lens 20 is provided with no aperture. The lens 20 can be assembled into the frame 10 and then surrounded by the inner ring surface 11 of the frame 10. The non-porous rim portion 21 of the lens 20 will be clamped by the convex engaging teeth 13 to affix the lens 20 into the frame 10. In other words, after the lens 20 is assembled into the frame 10, the lens 20 can be held within the frame 10 through the clamping between the convex engaging teeth 13 and the non-porous rim portion 21 and will not be easily separated from the frame 10.

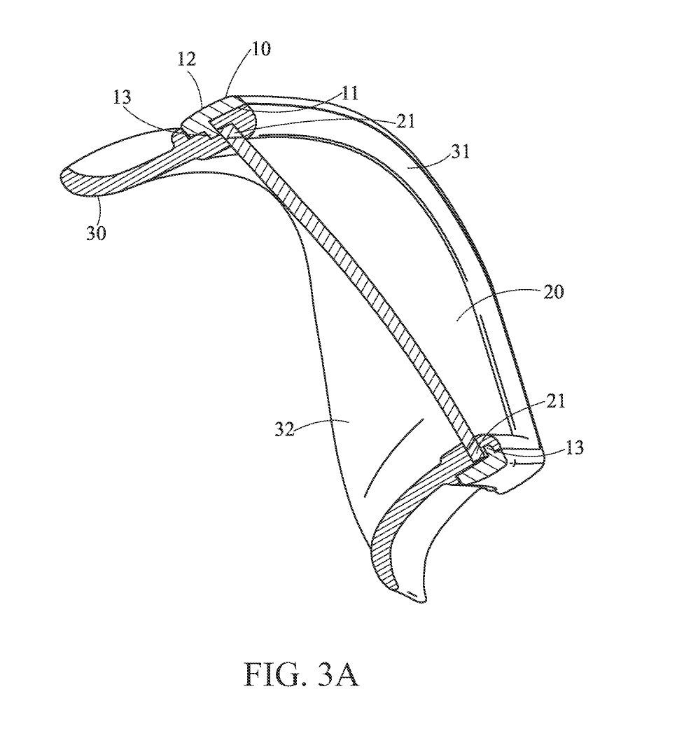

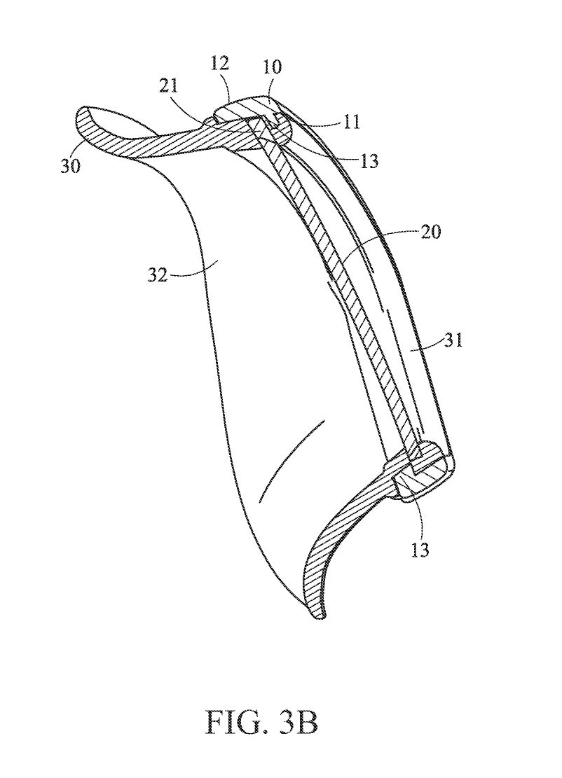

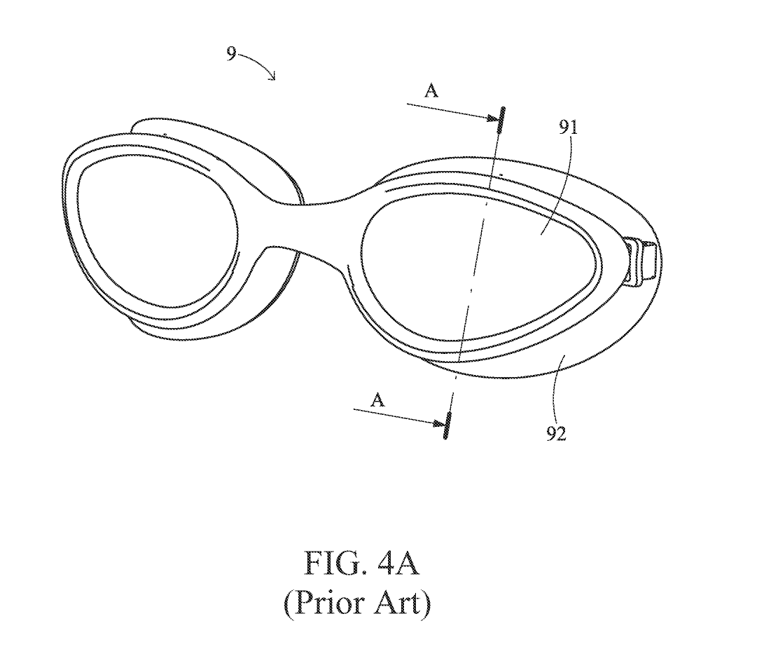

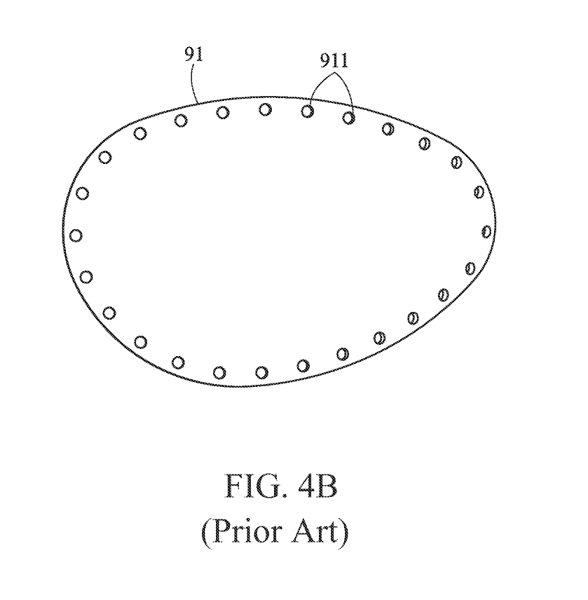

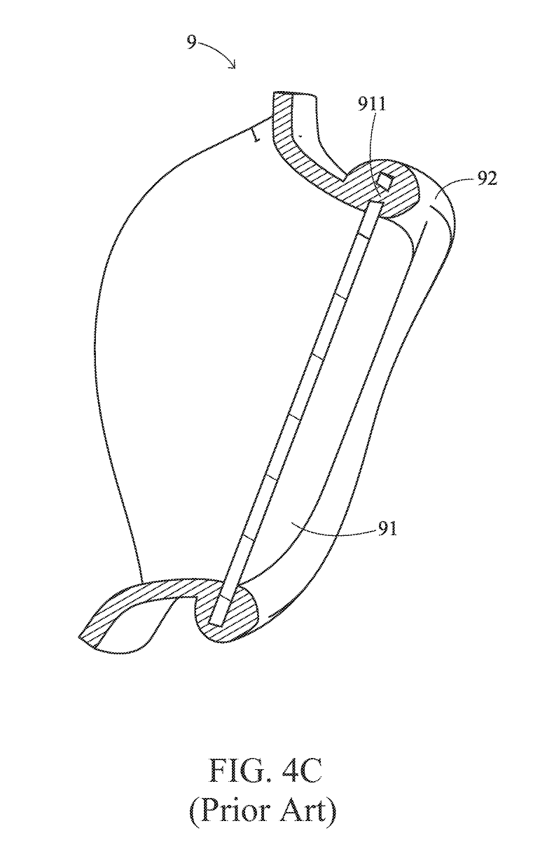

Tiny clearances may exist between the non-porous rim portion 21 of the lens 20 and the inner ring surface 11 of the frame 10, but these clearances will be filled with supporting structures 30. With reference to FIGS. 3A and 3B, a supporting structure 30 specifically comprises a covering portion 31 and a supporting portion 32. The covering portion 31 may encapsulate the inner ring surface 11 and the convex engaging teeth 13 of the frame 10 as well as the non-porous rim portion 21 of the frame 20 to render a gap between the inner ring surface 11 and the non-porous rim portion 21 waterproof In other words, the covering portion 31 blocks the potential clearances between the inner ring surface 11 and the non-porous rim portion 21 so that water or dust cannot pass therethrough. That is to say, the connection between the inner ring surface 11 and the non-porous rim portion 21 is a waterproof connection due to the encapsulation of the covering portion 31. Optionally, the covering portion 31 may further encapsulate the outer ring surface 12 of the frame 10 (not shown).

The supporting portion 32 of the supporting structure 30 extends backwards from the covering portion 31 and is attached to the skin around the user's eyes so that water or dust cannot pass through the space between the supporting portion 32 and the skin. The supporting portion 32 and the covering portion 31 may be formed integrally or be bonded together through multiple injection molding so that no clearance exists therebetween. The connection piece 40 connects the two supporting structures 30 and may be formed integrally with the covering portion 31.

What is described above illustrates the structures of the elements of the waterproof goggle 1. The materials for manufacturing the aforesaid elements may be as follows (not excluding other possible materials): the material for manufacturing the lens 20 may include cellulose acetate, cellulose biacetate, cellulose triacetate or polycarbonate; the material for manufacturing the supporting structure 30 may be a soft material including thermoplastic rubber (TPR), thermoplastic polyurethane (TPU), thermoplastic elastomer (TPE), polyvinyl chloride (PVC), silicone rubber or natural rubber; and the material for manufacturing the frame 10 may include polycarbonate, polyoxymethylene, acrylonitrile butadiene styrene, nylon or polypropylene.

The number of the elements comprised in the waterproof goggle 1 may also be adjusted. For example, the waterproof goggle 1 may be adjusted to comprise a single frame 10, a single lens 20 and a single supporting structure 30 (in this case, the connection piece 40 becomes unnecessary) or comprise two frames 10, two lenses 20 and a single supporting structure 30, thereby constructing various types of waterproof goggles. In other words, as to the number of the elements, the waterproof goggle 1 may be defined to comprise at least one frame 10, at least one lens 20 and at least one supporting structure 30 and optionally comprise the connection piece 40.

In terms of the technical effect, the lens 20 of the waterproof goggle 1 is not perforated, so the covering portion 31 of the supporting structure 30 does not need to additionally block the perforations; thus, the use of the material for the covering portion 31 may be reduced. Additionally, the covering portion 31 only needs to cover the potential clearances between the lens 20 and the inner ring surface 11 of the frame 10, so the thickness of the covering portion 31 on the inner ring surface 11 may be relatively small, i.e., the frame 10 may still be thin after the covering portion 31 is added thereto. As the frame 10 is thin, resistance to the flow of water cause by the frame 10 is relatively small.

The above disclosure is related to the detailed technical contents and inventive features thereof People skilled in this field may proceed with a variety of modifications and replacements based on the disclosures and suggestions of the invention as described without departing from the characteristics thereof Nevertheless, although such modifications and replacements are not fully disclosed in the above descriptions, they have substantially been covered in the following claims as appended.

What is claimed is:

1. A waterproof goggle, comprising:
a frame, being a ring structure and having an inner ring surface, an outer ring surface and a plurality of convex engaging teeth, wherein the inner ring surface is spaced apart from the outer ring surface and the convex engaging teeth are disposed on the inner ring surface;
a lens, being surrounded by the inner ring surface of the frame and having a non-porous rim portion which is clamped by the convex engaging teeth to affix the lens into the frame; and
a supporting structure, having a covering portion and a supporting portion which extends backwards from the covering portion, wherein the covering portion encapsulates the inner ring surface, the convex engaging teeth and the non-porous rim portion to render a gap between the inner ring surface and the non-porous rim portion waterproof.

2. The waterproof goggle of claim 1, wherein any two neighboring ones of the convex engaging teeth are staggered in a front-back direction.

3. The waterproof goggle of claim 1, wherein each of the convex engaging teeth has a shape and the shapes of the convex engaging teeth are different from one another.

4. The waterproof goggle of claim 1, wherein the covering portion of the supporting structure further encapsulates the outer ring surface of the frame.

5. The waterproof goggle of claim 1, wherein the frame, the lens and the supporting structure are configured to be two frames, two lenses and two supporting structures respectively, and the waterproof goggle further comprises a connection piece which connects the two supporting structures.

6. The waterproof goggle of claim 1, wherein material for manufacturing the lens includes cellulose acetate, cellulose biacetate, cellulose triacetate or polycarbonate.

7. The waterproof goggle of claim 1, wherein material for manufacturing the supporting structure is a soft material including thermoplastic rubber, thermoplastic polyurethane, thermoplastic elastomer, polyvinyl chloride, silicone rubber or natural rubber.

8. The waterproof goggle of claim 1, wherein material for manufacturing the frame includes polycarbonate, polyoxymethylene, acrylonitrile butadiene styrene, nylon or polypropylene.
